(19) United States
(12) Patent Application Publication
(10) Pub. No.: US 2018/0221276 A1
(43) Pub. Date: Aug. 9, 2018

(54) WELLNESS DRINK AND METHOD OF MANUFACTURE

(71) Applicants: Chris V. Malham, Scottsdale, AZ (US); Natalie Dawn Malham, Scottsdale, AZ (US)

(72) Inventors: Chris V. Malham, Scottsdale, AZ (US); Natalie Dawn Malham, Scottsdale, AZ (US)

(73) Assignee: Drink NOVE, LLC, Phoenix, AZ (US)

(21) Appl. No.: 15/426,275

(22) Filed: Feb. 7, 2017

(51) Int. Cl. A61K 9/00; A61K 31/19; A61K 36/886; A61K 36/752; A61K 35/644; A61K 36/63; A61K 36/81; A61K 36/9068; A61K 36/54; A61K 36/9066; A23F 3/40; A23L 2/52

(52) U.S. Cl. CPC A61K 9/0095; A61K 31/19; A61K 36/886; A61K 36/752; A61K 35/644; A61K 36/63; A23V 2002/00; A61K 36/9068; A61K 36/54; A61K 36/9066; A23F 3/40; A23L 2/52; A61K 36/81

(57) ABSTRACT

A wellness drink is disclosed comprising a mixture of apple cider vinegar, aloe vera, lemon juice, honey, olive oil and at least two spices selected from cayenne pepper, cinnamon, turmeric and ginger. The wellness drink may be high pressure processed to cold pasteurize the drink and then refrigerated and bottled. The typical ten ounce serving for an adult may be taken in the morning with breakfast. The drink may be sold comprising the liquid and spice ingredients without the water to save weight and then mixed with pure water. If taken on a regular basis, the wellness drink may reduce abdominal pain, belching and bloating and induce weight loss and a general sense of well-being. The drink tastes like apple cider with added spices and has a dark yellow orange color. Other forms include capsule, a tea bag, a spice pack and a pill.

### TECHNICAL FIELD

The present embodiments relate generally to wellness drink compositions and to methods of making and using such compositions and, more particularly, to a wellness drink which has been demonstrated to improve abdominal discomfort, improve feelings of bloating, belching and increase weight loss.

### BACKGROUND

People of all ages, genders and races suffer at some point from various types of abdominal pain including bloating, gas pain and other forms of abdominal discomfort. The abdominal discomfort may be misdiagnosed by a medical doctor, for example, as H. pylori, a germ-infested stomach bacterium that may enter the body and live in the digestive tract.

Conventional treatment with prescribed and over the counter (OTC) drugs such as Pepto Bismol, Kaopectate or acetaminophen (Tylenol) or non-steroidal anti-inflammatory drugs (NSAIDs), and narcotics have remained the mainstay of current treatments. Some prescribed medications may cost on the order of hundreds of dollars per week in dosage costs. However, these treatments, themselves, are typically associated with significant adverse side effects (e.g., gastrointestinal, cardiovascular, and even addiction).

NSAIDs are among the most frequently used class of drugs worldwide, with yearly over-the-counter sales amounting to $30 billion. Gastrointestinal safety continues to be a high priority for patients and clinicians when choosing an NSAID treatment for pain. In fact, the gastrointestinal harm induced by NSAIDs may be the most prevalent adverse event associated with any drug class. Clinical manifestations of adverse gastrointestinal events include gastric and duodenal mucosal erosions, ulcers and ulcer complications, dyspepsia, abdominal pain and nausea. Dyspeptic symptoms include epigastric pain, bloating, nausea and heartburn, which account for the most common reason for discontinuation of NSAID therapy. Gastric or duodenal ulceration occurs in about 20% of NSAID users, and 40% of these individuals develop a serious complication. Other problems in the lower gut linked to the use of NSAIDs are gut inflammation, increase in gut permeability, stricture, protein malabsorption, bleeding, and perforation. Therefore, as a result of the widespread use of these agents, the potential for a significant number of adverse events, particularly gastrointestinal related, is high. Gastrointestinal adverse events associated with NSAID use are reported to account for more than 100,000 hospitalizations and more than 15,000 deaths annually. Noteworthy are the numbers of hospitalizations for patients taking long-term, low-dose aspirin who are admitted with upper gastrointestinal bleeding. This accounts for about 10-15% of the hospital admissions for upper gastrointestinal bleeding. The resulting economic costs incurred in managing NSAID related gastrointestinal adverse events are significant; where it is estimated that $0.66-1.25 of every dollar spent on the cost of the NSAID is associated with treating adverse events.

Selective (COX-2) inhibitors have demonstrated improved gastrointestinal tract safety over traditional NSAIDs drugs. There is important evidence from clinical trials showing that compared with traditional NSAIDs, COX-2 inhibitors are associated with a reduced rate of serious GI events such as bleeding, perforation and obstruction, and other symptoms such as dyspepsia, as well as a reduced requirement for concomitant gastroprotective therapies such as proton pump inhibitors. This relative benefit may be related to a lack of COX-1-mediated inhibition of gastric mucous production and a lack of effect on platelet thromboxane production. However, the differential effects of COX-2 inhibitors compared with traditional NSAIDs on platelet aggregation, prostacyclin/thromboxane balance, and inflammatory mediators involved in the development of atherosclerosis have also led to concerns that there is a physiological basis for COX-2 inhibitors to increase the risk for thrombotic events. These negative cardiotoxic effects (myocardial infarctions) of the COX-2 inhibitors were first documented in the Vioxx Gastrointestinal Outcomes Research (VIGOR) trial and the Celecoxib Long-term Arthritis Safety Study (CLASS). Although the cardiotoxic effects were thought to be limited to myocardial infarctions, a subsequent meta analysis showed an increase in the occurrence of arrhythmias in COX-2 treated patients as well. The ensuing body of evidence relating to adverse cardiovascular outcomes prompted the FDA to remove rofecoxib (Vioxx®) from the market and led to modified warnings and use of Celecoxib (Celebrex®). Additionally, resulting changes to pain treatment recommendations have led to a significant decline in the use of the COX-2 inhibitors.

Because of the widespread use of NSAIDs and COX-2 inhibitors, the risks associated with their use are of increasing concern. In the recently concluded 2009 American Geriatrics Society (AGS) annual meeting; as a result of their troubling side effect profiles, the revised AGS guidelines on the management of persistent pain to be published in the August issue of the Journal of the American Geriatrics Society adopted the position and will advise physicians to have their elderly patients avoid the use of NSAIDs and COX-2 inhibitors and consider the use of low-dose opioid therapy instead. This position reflects general safety concerns with the use of these agents.

As the population ages and even young patients are experiencing weight gain for lack of nutritional fresh vegetables and fruits. Children are encouraged to eat and a number of documentaries have reinforced the position that processed foods are causing excessive weight gain and gastrointestinal pain at all ages. More and more patients are undergoing visits to detoxification centers just to cleanse their intestinal tracts at high costs including their living costs at such facilities. As a result, wellness drinks or drinks purporting to promote wellness are increasing in quantity and variety. While the young and poor are at risk when they are served lunches full of sugar and high levels of fatty acids and cholesterol through school lunch programs, these children have a need for non-processed food or high quality food such as fruits and vegetables that are natural, organic and safe.

Wellness drinks and tonics have been known since the traveling road druggists of the 1800's. Most of these drinks and tonics contained alcohol. Recently, there has been renewed interest in wellness drinks. These drinks are not harmful such as those containing alcohol but have not been demonstrated to promote wellness. There, therefore, remains a need for effective, but safer alternatives for the treatment of abdominal pain, bloating and indigestion caused by eating processed foods and other causes.

Many anecdotal as well as recent studies support the use of natural remedies (herbal, vegetable and fruit) for relief of pain. Historically herbal remedies have not only been reported as effective as drugs, but they have been used to treat various ailments and conditions and generally have had very low risk profiles. Such existing remedies may not typically be as effective as pharmaceutical abdominal pain relief products that are currently available OTC or by prescription.

### SUMMARY

Example embodiments of a wellness drink according to the present invention are generally directed to compositions that include natural ingredients for the treatment of abdominal discomfort in mammals. The wellness drink of the present invention avoids the use of alcohol and any form of over-the-counter or prescription drugs. In particular, according to non-limiting example embodiments, compositions provided herein may include anywhere from four to ten ingredients. In a first recipe, the main ingredients include citric and/or acetic acids and more neutral pH ingredients that are natural and organic. For example, a first recipe may comprise pure (for example, boiled) water mixed with apple cider vinegar, lemon juice from one or more freshly squeezed lemons, aloe vera juice or gel made from the aloe vera leaf or stem, honey, for example, clover honey, olive oil or coconut oil and additive medicinal powders and spices such as turmeric, ginger, cinnamon and cayenne pepper.

Now, the benefits of certain of the ingredients will be discussed. Apple cider vinegar (acetic acid) is a mild acid that further contains minerals, vitamins and enzymes that balance pH levels of a mixture of component ingredients toward a lower value than would be expected by chemists regarding the chemical mixture alone without knowing quantities. Apple cider vinegar may reduce cholesterol, internally cleans a human system, provides energy, reduces bloating, lessens water weight and is an appetite suppressor. Aloe vera juice or gel aids in digestive health, balances stomach acids, assists the body to absorb consumed nutrients and has laxative qualities. Aloe vera contains an anti-inflammatory glycoprotein responsible for healing internal wounds which may hinder digestion and so has anti-inflammatory, antioxidant and free radical scavenging properties which may help humans suffering from arthritis and joint pain. Freshly squeezed lemon juice (citric acid) contains vitamins such as vitamin C and nutrients. It is a mild citric acid which can lower Ph and is an anti-oxidant. Lemon juice is a source of potassium and electrolytes, more than either apples or grapes and are high in calcium which helps strengthen bones and teeth. Honey, for example, clover honey is credited with anti-aging properties, antibacterial, anti-inflammatory anti-bacterial and anti-oxidant properties. Honey is easy to digest. Honey is known to help prevent the risk of stroke and arteriosclerosis, regulates blood pressure, controls liver problems and stimulates bowel activity. Clover honey, not subjected to heat, can improve bad cholesterol levels. Olive oil, for example, extra virgin olive oil, is anti-inflammatory, an anti-oxidant, and contains heart-healthy nutrients. Olive oil is known to lower rates of heart disease, depression and dementia, encourages weight loss and helps to prevent diabetes.

The spices comprise cayenne pepper, ginger, turmeric and cinnamon. Cayenne pepper breaks up mucus, unblocks clogged arteries, encourages weight loss and may curb a human appetite, reduce bloating, speed up metabolism and burn calories. Cayenne pepper increases blood flow, increasing sex drive, reduces ulcers and promotes healthy digestion. Ginger alleviates symptoms of gastrointestinal distress. Ginger is a “carminative” which relaxes and smooths the intestinal tract. Ginger has anti-oxidant effects, inhibiting the formation of inflammatory compounds. Turmeric is an anti-inflammatory agent for treating flatulence, jaundice, menstrual difficulties, bloody urine, hemorrhage, toothache, bruises, chest pain and colic. Cinnamon has antioxidant and anti-inflammatory properties reducing the risk of heart and neurodegenerative diseases. Cinnamon lowers blood-sugar levels, has an anti-diabetic effect and may be a protection against cancer. Cinnamon fights bacterial and fungal infections and may prevent tooth decay and reduce bad breath.

Also, for production purposes, it is suggested that high pressure processing (HPP) equipment, for example, equipment available from an international corporation known as Hyperbaric with a US subsidiary: Hyperbaric USA, 2250 NW 84th Avenue, Unit 101, Miami, Fla., be used to cold pasteurize the finished mixture removing any bacteria and potentially causing some chemical reactions among one or more ingredients to produce some chemicals that are not found in nature. HPP provides cold pasteurization and subjects a wellness composition to pressures between 43,500 psi and 87,000 psi.

According to non-limiting example embodiments, the present compositions help provide general gastrointestinal relief of the pain associated with stomach bloating, gas pains, menstrual cramps, and other sources of abdominal pain as well as promote a cleansing and a possible weight loss.

Example compositions according to the present application are believed to be safer, more potent, and provide effective, lasting relief of pain from many conditions than any prescription medications and may be taken typically in the morning once before the start of the day as one would choose in the alternative to drink a glass of orange juice.

According to other non-limiting example embodiments, methods are provided, which include administering an effective amount of the present compositions to a mammal in need thereof. The typical bottle of the nine or ten ingredient products may be sized at from six to twelve ounces and preferably comprises a ten ounce bottle (or can). In weight, pure water may comprise approximately 150 to 200 grams, for example, 178 grams by weight out of 296 grams total. Consequently, it is also an embodiment of the present invention that may comprise a pouch or other container of all ingredients except the water, namely, the vinegar, lemon juice, aloe vera, honey, olive oil and four spices that have a mass of only 118 grams versus 296 grams mass when water is included. This embodiment or mixture with water to be added may also be subjected to HPP and stored refrigerated in a refrigerator section of a food or drug store. It may be purchased in a pouch or other container and mixed with cool pure water saving shipment to a store of 178 grams of water, for example, as a six-pack of pouches kept in a refrigerator or a twelve-pack.

It is also contemplated to form a mixture of spices alone for seasoning meats and vegetables and to make a hot liquid tea with a tea bag comprising a mixture of spices. Further potential embodiments of the wellness drink may comprise some or all of the ingredients formed into a pill or capsule, for example, as a once-a-day pill or capsule similar to a vitamin. Dry citric acid is known as is organic aloe vera powder. Drying acetic acid (vinegar) may be possible in the future to remove the water. Dry honey powder is known. Olive oil may be concentrated into a wax and used as a pill coating or used in a capsule form. Consequently, there may be a number of different embodiments of the present wellness drink in many forms as will be explained further herein.

Further provided are methods of making the compositions provided herein.

### BRIEF DESCRIPTION OF THE FIGURES

Embodiments of the invention are herein described, by way of non-limiting example, with reference to the following accompanying Figures:

FIG. 1 is a wellness drink recipe showing a table of volume in ounces versus ranges of a wellness drink for a portion between six and thirty ounces;

FIG. 2 is a further production formula showing the ingredients percent compositions as a range including a preferred value and the value of grams of the identified ingredient;

FIG. 3 is a flowchart showing a typical high pressure processing where a very high pressure applied to a mixture of ingredients is added to pure water and then succumbs to a pressure which may reach a level of 87,000 psi. Such a pressure is known to perform a cold pasteurization of the mixture, the final step being to unload the wellness drink product and bottle the product, for example, in single serving bottles between six and twelve ounces.

FIG. 4 is a black and white drawing of apparatus 400 showing the measurement of pH of a first prototype production sample at 3.42. As described herein, the pH appears to be within a range of 3.07+ or −0.20.

FIG. 5 is a black and white drawing of high pressure processing equipment 500 which exemplifies the process shown in FIG. 3 where a container of ingredients in placed on a first platform 510, water is introduced from a tank, the mixture of ingredients is placed under extreme pressure exhibited by the pressure tank 520 and output to a final platform 530 having been cold pasteurized by the process. The high pressure will induce some chemical reactions among the ingredients: for example, the acids, the honey and the aloe vera.

These and other features of the present wellness drink will be described in the DETAILED DESCRIPTION which follows.

### DETAILED DESCRIPTION

Generally provided herein are various compositions and methods that provide general analgesic relief of abdominal pain and for the treatment of weight gain in mammals. Present embodiments include methods of treating or preventing abdominal pain caused e.g., from eating processed foods and generally eating or drinking to excess, including stomach bloating, belching, abdominal pain, gas pain and other abdominal pain. Also included are methods of making the wellness drink compositions herein for commercial production.

Various combinations of natural products for wellness have been tried in the past, but the potency and effect of such products was not sufficient to replace prescription or over-the-counter (“OTC”) digestive improvement products that are synthetically derived in a laboratory. The present inventors discovered that particular combinations of ingredients have unexpectedly superior abdominal pain relieving effects and provide for varying degrees of intestinal tract cleansing and weight loss. The potency of the present compositions is much higher for the general relief of abdominal pain than that of prior over-the-counter products and prescription medications. Therefore, the present compositions can be safely used as a substitute for synthetic abdominal pain relievers, while retaining the safety of a natural product. The only expected “synthesis” is any chemical reactions that may be caused during high pressure processing (HPP) which performs a cold pasteurization.

The aspects, advantages and/or other features of example embodiments of the invention will become apparent in view of the following detailed description, taken in conjunction with the accompanying drawings. It should be apparent to those skilled in the art that the described embodiments of the present invention provided herein are merely exemplary and illustrative and not limiting. Numerous embodiments of modifications thereof are contemplated as falling within the scope of the present invention and equivalents thereto. Any publications, patent applications, patents, and other references mentioned herein are incorporated by reference in their entirety.

In describing example embodiments, specific terminology is employed for the sake of clarity. However, the embodiments are not intended to be limited to this specific terminology.

As used herein, “a” or “an” may mean one or more. As used herein, “another” may mean at least a second, third, fourth or more and so on. Furthermore, unless otherwise required by context, singular terms include pluralities and plural terms include the singular.

As used herein, “composition”, “therapeutic composition”, “recipe”, and “formulation” may be used interchangeably and refer to a combination of elements that is presented together for a given purpose. Such terms are well known to those of ordinary skill in the art.

As used herein, the term “core ingredient” is intended to encompass those ingredients of the present composition that are the most important in the present compositions and methods. According to non-limiting example embodiments, compositions provided herein include at least the following five core ingredients, apple cider vinegar, lemon juice, aloe vera liquid or gel, honey, olive oil, and a medicinal mixture of spices which may comprise cayenne pepper, turmeric, ginger and cinnamon. According to other example embodiments, “core ingredients” may include or exclude one or more additional or different ingredients than those listed above, such as cinnamon or one or more synergistic ingredients or other natural ingredients that may be used to treat a mammal in need of treatment.

As used herein, the term “synergistic ingredient” is intended to encompass natural ingredients that are not necessarily “core ingredients” in the present compositions, but may be added or subtracted from the present compositions for example to add to the abdominal pain relief that may be achieved by the compositions, or to add to the synergistic effects to be achieved by the present compositions, or to improve efficacy, etc. Non-limiting example embodiments of possible synergistic ingredients include one or more ingredients selected from cinnamon and ginger.

The terms “drug” and “active ingredients” are used herein to include any drug or other active ingredient that may be added or caused by HPP processing of the ingredients when placed under high pressure pasteurization and refrigeration in addition to the core and synergistic ingredients for treating mammals for a variety of different abdominal and other conditions. Such drugs or active ingredients are not intended for production or for commercial sale and promotion but may be appropriately added for relief of particular stomach/abdominal conditions.

By way of non-limiting example embodiment, additional core ingredients or other drugs or active ingredients that may be added to the present compositions may include the addition of Green Tea, and possibly Fish Oil and certain so-called Essential Fatty acids, calcium and/or other minerals. These terms are not meant to be limiting and may include any “active ingredient” and “drug” known to those skilled in the art, which may be incorporated in the formulations herein.

The terms “core ingredients,” “synergistic ingredients,” and “active ingredients” and “drugs” are intended to encompass such ingredients in all forms including, but not limited to extracts, powders, analogs, waxes, prodrugs, salts and/or crystalline forms thereof as would be apparent to those skilled in the art.

The term “excipient” is used herein to include pharmaceutically acceptable inert substances added to a drink formulation to give e.g., a desired consistency or form, or used as a carrier. Non-limiting examples of excipients that may be included in the present compositions and/or formulations herein may include, but are not limited to binders, fillers, diluents, lubricants, disintegrants, super-disintegrants, and other excipients known to those skilled in the art, depending e.g., on the composition being formed, method of formation, active ingredient(s) being used, etc.

Recommended Dosage and Use

A recommended dosage is for an adult to consume a ten ounce bottle each morning. However, dosage levels for younger or thinner patients may be reduced to, for example, six or eight ounces and the bottled product may be sold in quart, liter, half-liter or other sized bottles, stored refrigerated and poured into an appropriately sized container for consumption.

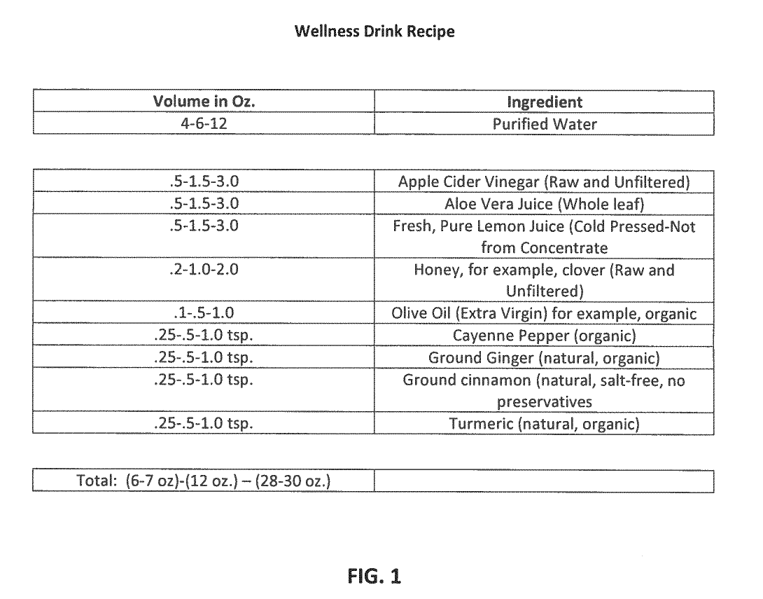

Referring now to FIG. 1, there is shown a representative recipe for a wellness drink which at least induces abdominal health and is shown comprising a range of values with the center value, in ounces or teaspoons (the spices) being a preferred value. The claims are not intended to be limiting but may include fewer or greater numbers of ingredients than the nine shown and olive oil in an early prototype was replaced with coconut oil. The coconut oil was not found to comprise an ingredient that could be utilized in a product due to physical and chemical changes caused by HPP. On the other hand, a recipe may include coconut oil if the recipe is made at home or in a laboratory or factory without HPP processing.

The values of total ounces show a range of values that may be used for a smaller portion such as six or eight ounces or a larger portion such as two pints or one quart of thirty-two ounces. Larger or smaller quantities may be sold and maintained refrigerated until used. The value in the center was the first value selected for dosage at twelve ounces.

The initial recipe comprises purified water; apple cider vinegar (preferably raw and unfiltered); aloe vera juice or gel (for example, made from the whole leaf or stems of an aloe vera plant); freshly squeezed lemon juice (for example, cold-pressed and not from concentrate which has been processed); and olive (or coconut oil) where the olive oil is preferably extra virgin olive oil and organic. Four spices are shown in this initial recipe comprising cayenne pepper, ground ginger, cinnamon and turmeric each in quantities of one-half teaspoon for a twelve ounce serving. Preferred quantities for a twelve ounce morning serving for an adult are six ounces of purified water, one and one half ounces of each of apple cider vinegar, aloe vera, lemon juice and one ounce of honey, preferably clover honey.

Elimination of the water from the wellness drink mixture permits one to purchase a container saving a considerable volume of water and so may be mixed at home with an appropriate volume of pure water, for example, once per day. Other forms of the wellness drink will be discussed herein including a tea, a capsule and a pill form.

Production of a Prototype Wellness Drink

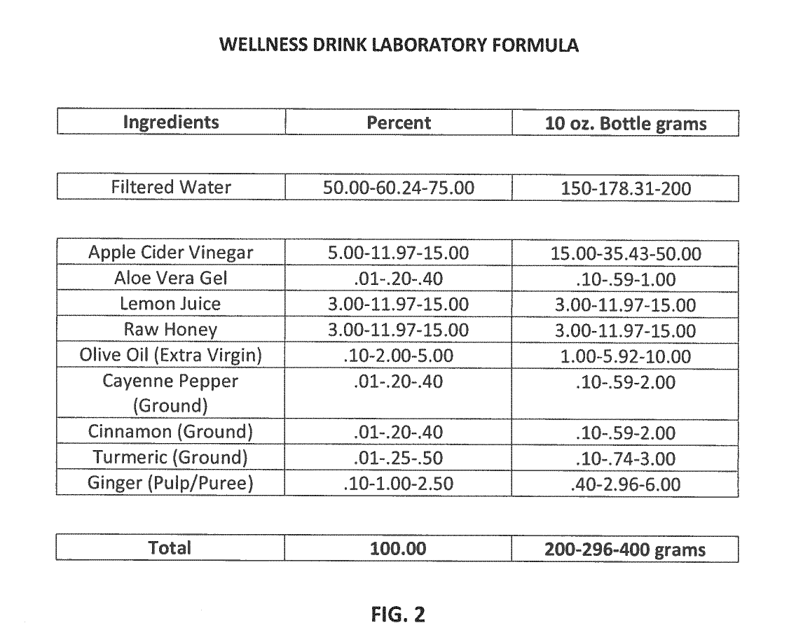

Referring now to FIG. 2, there is shown a table showing a typical composition of a ten ounce adult serving comprising most of the original ingredients of the original recipe. Again, there are shown ranges of values with the center value (the value being the center value of three values forming a range of values) being a preferred value for commercial production. Not all values of the ranges are discussed below but are clearly seen in FIG. 2. The values (range or preferred) of each ingredient are shown in percent and in grams per ten ounce serving. Filtered or other water, for example, spring water, preferably comprises 60.24% of the volume and 178.31 grams; apple cider vinegar comprises 11.97% and 35.43 grams, aloe vera gel (replacing juice) may comprise preferably 0.20% by volume and 0.59 grams; lemon juice (freshly cold squeezed) and raw honey, for example. clover honey, may comprise the same 11.97% by volume which also comprises the number of grams at 11.97 grams; and extra virgin olive oil may comprise 2% by volume and 5.92 grams.

There are four spices in the preferred wellness drink product for commercialization: these are cayenne pepper (ground) and cinnamon (ground) at a preferred value of 0.20% each and 0.59 grams; turmeric (ground) is at a preferred value of 0.25% and 0.74 grams; and ginger made as a pulp or puree is at a preferred value of 1.00% and 2.960 grams. A ten ounce serving (the bottle should be refrigerated until used and after if the entire bottle is not consumed at one time) comprises two hundred ninety-six grams as a preferred value within a range of two to four hundred grams. Subsequent to FIG. 1 showing a twelve ounce serving, a ten ounce morning serving appeared to be preferable, and is shown in plans for making product, FIG. 2.

Eliminating the water from the wellness drink may form a mixture of ingredients which may, in turn, be preserved refrigerated and then mixed with an appropriate amount of pure water before drinking. Shipping of 178 grams of water per serving is saved per FIG. 2 for a pouch or other container which will make a ten ounce serving when mixed with the container of other ingredients. The remaining amount of ingredients weigh only 118 grams per ten ounce serving and may be sold as a six pack or a twelve pack in a store. HPP is recommended for the “pouch” embodiment to cold pasteurize the ingredients followed by refrigeration for preservation.

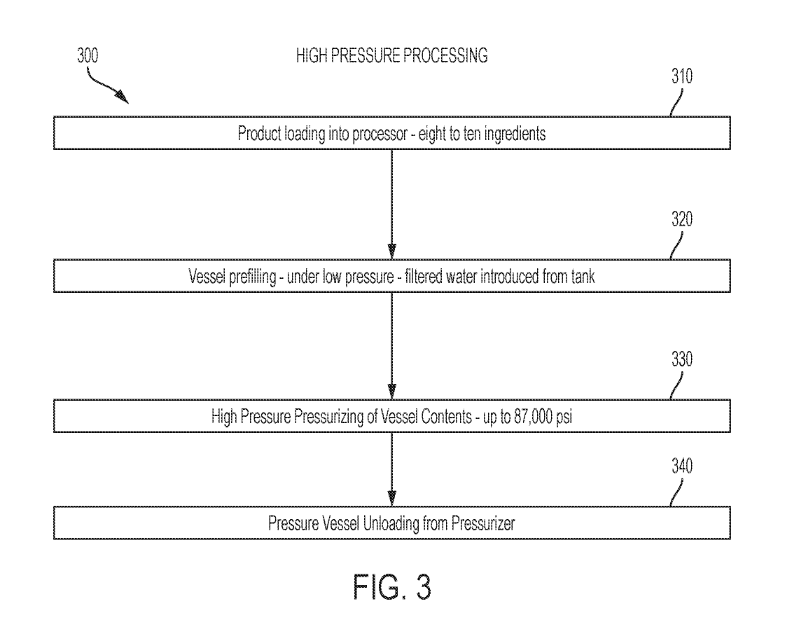

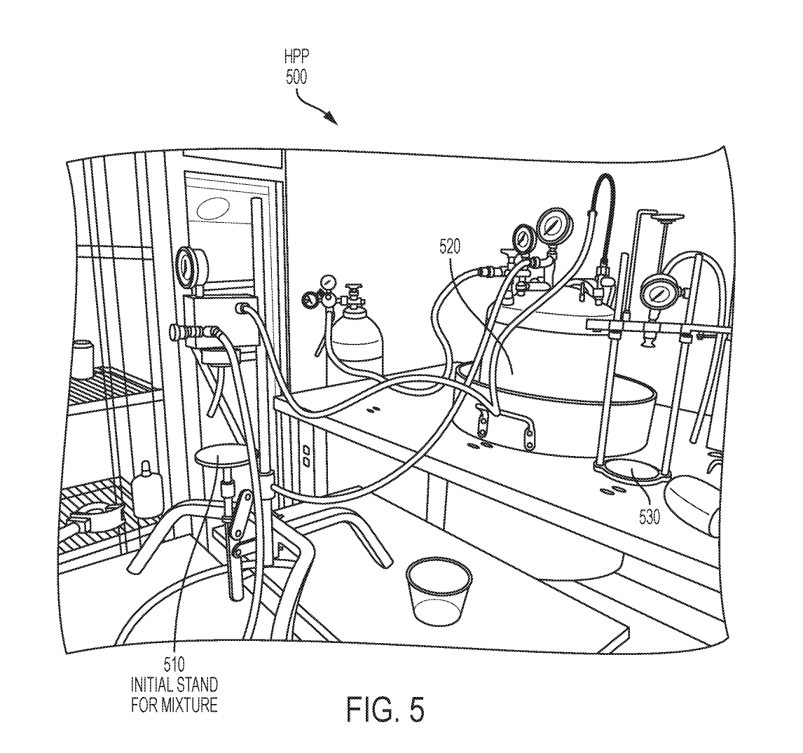

A flowchart of high pressure processing for cold pasteurization 300 is shown in FIG. 3. A mixture of ingredients (except water) is formed and loaded into a staging area at step 310 of HPP process 300 using the HPP equipment, for example, from Hiperbaric USA as seen in FIG. 5. As discussed above there may be from eight to ten ingredients preferably or between five and twelve ingredients depending on the above-discussed possible additional ingredients depending on a particular application. At step 320, filtered, spring or other clean water is introduced into a container vessel at step 320, still in the staging area of FIG. 5. The contents of the container vessel are then transferred for HPP processing to a large pressure tank with pressure gauges as seen in FIG. 5. HPP subjects a mixture of an embodiment to between 43,500 psi and 87,000 psi with an average pressure of about 60,000 psi. Step 330 shows the pressurization process itself and shows that Hyperbaric equipment may apply a pressurization (cold) at between forty and eighty-thousand pounds per square inch to the water mixture (or mixture without water added) in the container vessel. At step 340 the pressure vessel is unloaded and the pressurized mixture is unloaded and deposited, for example, in a ten ounce serving bottle or a pouch (without water added) or other container and refrigerated for preserving freshness.

Testing for pH Level and Sugar Content

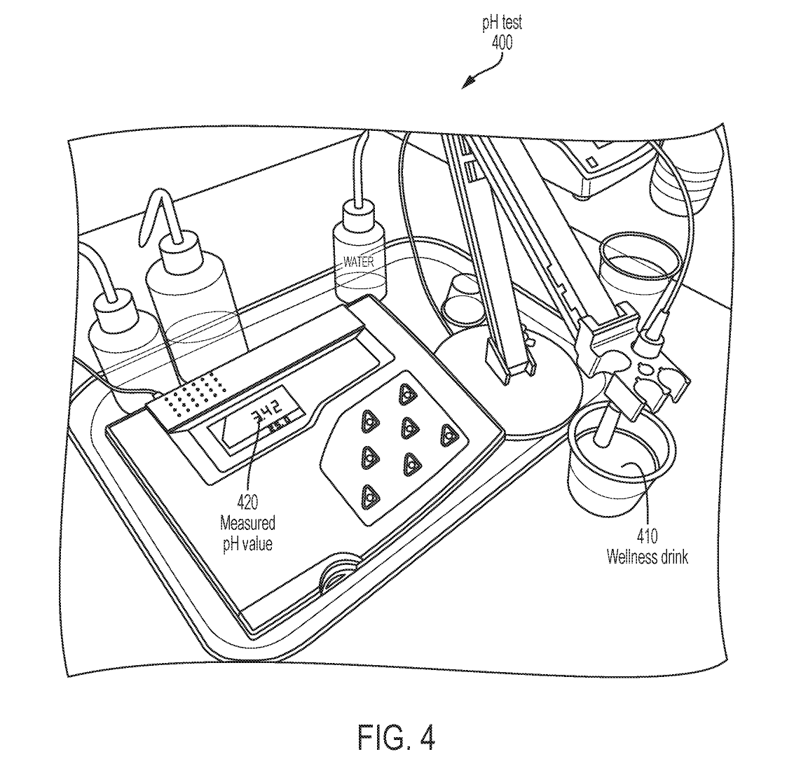

Ideally for commercialization, a wellness drink should have a low or more acidic pH level, for example, with a range of 2.5 to 3.75. Clearly, one does not want to have an even lower value which can cause acid indigestion. The preferred mixture after HPP processing was tested for pH level and has been retested several times such that, referring to FIG. 4, the initial prototype was tested and exhibited a pH level of 3.42. Upon further testing, it was determined that the range of pH values for the wellness tonic were 3.07+ or −0.20.

The Food and Drug Administration requires that all labels showing ingredients of a canned or bottled food or drink comprise a nutrition label. For certain ingredients values are shown for those ingredients, except sugar content which can include additional sugar content added during food processing where the actual value of sugar must be shown whether natural or added to the food or drink. Sugar content of an aqueous solution is known in the art as a brix value in degrees. The range of Brix values for the wellness drink of the present invention was measured at 12.3°+ or −0.20 Bx. There is no sugar added to this drink—simply the sugar content found in the ingredients such as the apple cider vinegar and the lemon juice.

A table is shown below for the nutrition label for the present wellness drink. A range of values is not shown except for a wellness tonic ten ounce serving as refrigerated and maintained:

### Nutrition Facts

| | |
|---|---|
| Calories | 180 |

| | | | |
|---|---|---|---|
| Total Fat | 5 | grams | 8% |
| Saturated Fat | 1 | gram | 4% |
| Sodium | 5 | mg | 0% |
| Total Carbohydrates | 32 | grams | 12% |
| Total Sugars | 28 | grams | |
| Protein | 5 | grams | |
| Calcium | 31 | mg | 2% |
| Vitamin A | 75 | mcg | 8% |
| Vitamin C | 12 | mg | 15% |
| Vitamin E | 1 | mg | 6% |

The values for calcium through Vitamin E to the right in percent represent the percent minimum daily requirement (mdr) for the given nutrient.

The taste of the wellness drink, if maintained in a refrigerated state before drinking, is akin to apple cider with spice. Its natural color, if refrigerated as recommended, is a dark yellow orange.

Example embodiments as discussed above are also directed to methods of making the compositions or formulations herein. Such methods may include known HPP as well as other methods, especially refrigeration once the wellness drink or drink less water is produced.

A tablet may be made by compression or molding, optionally with one or more accessory ingredients. Liquid components must be dried and maintained refrigerated, for example, lemon juice or lemon peel or dried apple may be used in a capsule or pill. Dry citric acid is known but is missing the taste of lemon juice. Similarly, dry organic aloe vera powder is known for making a pill or capsule. Olive oil may be condensed and made into a form of wax suitable for forming into capsule form or as a coating for a pill. Dry honey powder is known. Drying acetic acid forms a crystalline substance but lacks the apple taste. Thus, once a day pills or capsules are possible but the wellness drink composition with or without water added is preferred.

Liquid preparations for oral administration may take the form of, for example, solutions, syrups or suspensions, or they may be presented as a dry product for reconstitution with water or other suitable vehicles before use. Such liquid preparations may be prepared by conventional means with pharmaceutically acceptable additives such as suspending agents (e.g., sorbitol syrup, methyl cellulose, or hydrogenated edible fats); emulsifying agents (e.g., lecithin or acacia); non-aqueous vehicles (e.g., extra virgin olive oil, aloe vera juice or honey in greater quantity may be used but is not as preferred as filtered or spring water.

Also encompassed herein are methods of treating a mammal (including, but not limited to humans). Example methods include administering the wellness drink to a mammal in need thereof, an effective amount of a composition or formulation provided herein in liquid form on a daily basis, preferably as a morning ritual.

Such administration, for example in the case of a ten ounce bottle, is typically by oral administration to a mammal and may replace other breakfast juices such as orange, grapefruit or tomato juice. Other forms of administration known to those skilled in the art are contemplated, depending on the formulation. By way of non-limiting example embodiment, other potential methods of administration may include methods known to those skilled in the art including, but not limited to, intraperitoneally, intravenously, orally, subcutaneously, intradermally, transdermally (e.g. pain patch), intramuscularly, intravascularly, endotracheally, intraosseously, intra-arterially, intravesicularly, intrapleurally, topically, intraventricularly, or through a lumbar puncture (intrathecally).

Formulations, such as from a ten ounce serving bottle, may be used for treating mammals for a variety of different conditions. The bottle less the water can be prepared as a pre-mixture for adding pure water as discussed above as a pouch. The present embodiments are generally to be used for the symptomatic relief of abdominal pain, such as minor to moderate acute pain (such as toothaches, menstrual cramps, heart conditions, inflammation of the intestinal tract, gas pains, etc.). Thus, the wellness drink compositions herein should be taken as directed, for example, once per day, until the bloating, belching, over weight condition or other condition is relieved.

The natural and organic composition of this wellness drink invention may involve the chemical reaction of two or more ingredients. For example, while an acid will not react with another acid, it is conceivable that the lemon or apple cider vinegar ingredients which comprise mild acid pH levels may react with another ingredient including aloe vera, honey and the spices to form a new ingredient.

The present wellness drink compositions of the present invention are unlike any other wellness drink product on the market. A bottle, pouch, capsule, pill, spice pack, or tea bag may combine certain of the ingredients for the analgesic effect of natural anti-inflammatories and also support gastro-intestinal health. Example compositions according to the present application are believed to be safer, more potent, and provide effective, lasting relief of pain from many conditions including, for example, toothaches, menstrual cramps, stomach aches, bloating and belching, abdominal inflammation, chronic abdominal pain, etc.

Aloe vera is extracted from the aloe vera plant leaves and stems. Once an appropriate mixture of fresh leaves is obtained, the leaves of the mixture are then pulverized, in order to rupture the cells of the leaves, and the mixture is maintained for an amount of time sufficient to release intracellular material from the leaves and create a liquid from the leaf body or a gel from the aloe vera plant leaf stems. It should be understood that any method of pulverizing which physically ruptures the leaf cells, such as homogenizing, milling, grinding, chopping, blending, cutting, tearing, etc., may be used. A number of specific devices that may be suitably used to pulverized the aloe vera leaves in the leaf liquid or gel will be recognized by those of ordinary skill in the art, such as homogenizer, colloidal mills, stone mills, ball mills or tangential fluid energy mills. In accordance with the present method, the leaves in the aloe vera plant may be subjected to various degrees of pulverization. However, in one aspect, at least about 75% of all leaf cells may be ruptured. In another aspect, at least about 80% to about 99% of all leaf cells may be ruptured. Similar considerations should be given to ginger and making a puree or other liquid or powder of the ginger root.

The specific degree of pulverization, as well as other factors such as the exact type of plant used, the time of year at which the leaves or roots were harvested, and the amount of time that the leaf or root is maintained refrigerated following pulverization, will determine the efficiency of the overall extract process.

Over time, it has been observed that some of the components rise to the surface of the drink during refrigeration and other components precipitate to the bottom. During use, it may be appropriate to shake the drink to form a mixture of the components before drinking the wellness drink.

A spice pack may be used to flavor meats and vegetables from the four spices in combination.

Moreover, a tea bag may be formed of the spices and the powders of the typically liquid ingredients for making a hot or cold tea. It is contemplated that the wellness drink may be adapted for serving via a tea bag as is well known in the art. The tea bag is simple added to boiling water as well known and allowed to steep for, for example, a range of five to fifteen minutes, preferably, ten to twelve minutes. The recipe for a tea bag combination of spices is as follows for an eight ounce cup of tea:

| | |
|---|---|
| ¼ teaspoon | Turmeric |
| ¼ teaspoon | Ground ginger |
| ⅛^{th} teaspoon | Cayenne pepper |

It is suggested tat at organic raw and unfiltered honey such as clover honey be added to sweeten the hot tea which can be stirred into the hot tea. The amount of honey to be added is to one's taste but may comprise, for example, one teaspoon. Additional flavors of the tea may be obtained by adding dry powdered apple cider vinegar, cinnamon and/or fresh lemon juice or added lemon juice powder to the tea bag.

Although the wellness drink invention has been described in example embodiments and a method of processing disclosed, additional modifications and variations would be apparent to those skilled in the art. It is therefore to be understood that the inventions herein may be practiced other than as specifically described, for example with respect to the formulation type, portion size per day, use in the evening as well as in the morning or other time of day, etc. Thus, the present embodiments should be considered in all respects as illustrative and not restrictive. Accordingly, it is intended that such changes and modifications fall within the present invention as defined by the claims appended hereto.

1. A wellness drink comprising the following ingredients:
apple cider vinegar ranging between 0.5 and 3 ounces in quantity,
aloe vera ranging between 0.5 and 3 ounces in quantity,
freshly squeezed lemon juice ranging between 0.5 and 3 ounces in quantity,
honey from a bee hive ranging between 0.2 and 2 ounces in quantity, and
one of coconut and olive oil ranging between 0.1 and 1.5 ounces,
the wellness drink further comprising at least two of the following four spices: approximately 0.5 to 1 teaspoon of cayenne pepper, ginger, cinnamon and turmeric and mixed with pure water to make a six to twelve ounce serving.

2. The wellness drink of claim 1 further undergoing high pressure processing to cold pasteurize the wellness drink.

3. The wellness drink of claim 1 further comprising refrigeration during maintenance before drinking, the wellness drink for consumption once per day.

4. A wellness drink comprising the following ingredients
pure water comprising 150 to 200 grams,
apple cider vinegar comprising between 15 and 50 grams,
aloe vera juice or gel comprising 0.1 to 1 gram,
lemon juice comprising 10 to 50 grams,
honey comprising 10 to 50 grams,
extra virgin olive oil comprising 1 to 10 grams, and
four spices comprising cayenne pepper and cinnamon each comprising 0.1 to 2 grams, turmeric comprising 0.5 to 3 grams and one of ginger pulp or puree comprising 0.50 to 6 grams, the mixture being subjected to high pressure processing followed by refrigeration before consumption.

5. The wellness drink of claim 4 having a pH level within the range of approximately 3.0 plus or minus 0.2.

6. The wellness drink of claim 4 having a Brix sugar level approximately 12.0° plus or minus 0.2.

7. The wellness drink of claim 4 for reducing abdominal pain including at least two of bloating, belching, intestinal pain and gas pain.

8. The wellness drink of claim 4 wherein the honey comprises between ten and twelve grams of clover honey.

9. The wellness drink of claim 4 having a taste of apple cider with added spices.

10. The wellness drink of claim 4 having a dark yellow color.

11. The wellness drink of claim 4 wherein the drink specifically comprises a ten ounce liquid serving comprising approximately 180 grams of water, approximately 35 grams of apple cider vinegar, approximately 0.6 grams of aloe vera gel, approximately 35 grams of each of freshly squeezed lemon juice and honey, approximately six grams of olive oil, approximately 0.6 gm of each of cayenne pepper and cinnamon, approximately 0.7 grams of turmeric and approximately 3.0 grams of one of ginger pulp or puree to form an approximate serving size of 300 grams.

12. A method of manufacture of a wellness drink comprising
forming a mixture of apple cider vinegar, aloe vera, lemon juice, honey, olive oil liquids and four spices comprising cayenne pepper, ginger, cinnamon and turmeric, adding purified water,
measuring the p H of the mixture of water and formed mixture at 3.07+ or −0.20,
high pressure processing the water mixture of spices and the liquids comprising apple cider vinegar, aloe vera, lemon juice and honey,
depositing the processed mixture into a bottle having a serving size between six and twelve ounces and
maintaining the wellness drink bottles refrigerated until consumption by a user.

13. A method of making a wellness capsule comprising mixing dry citric acid, organic aloe vera powder, dry honey powder and mixing aloe vera wax with the dry ingredients within the capsule and further adding spices comprising cayenne pepper, ginger, cinnamon and turmeric.

14. A method of manufacture of a wellness drink comprising
forming a mixture of apple cider vinegar, aloe vera, lemon juice, honey, olive oil liquids and spices,
adding purified water,
high pressure processing the water mixture of spices and the liquids comprising apple cider vinegar, aloe vera, lemon juice and honey,
the added spices comprising at least three of cayenne pepper, ginger, cinnamon and turmeric,
depositing the processed mixture into a bottle having a serving size between six and twelve ounces and
maintaining the wellness drink bottles refrigerated until consumption by a user.

15. A method of manufacture of a wellness drink comprising
forming a mixture of apple cider vinegar, aloe vera, lemon juice, honey, olive oil liquids and four spices comprising cayenne pepper, ginger, cinnamon and turmeric,
high pressure processing the mixture of spices and the liquids comprising apple cider vinegar, aloe vera, lemon juice and honey,
depositing the processed mixture into a container for mixing with pure cold water, the mixture having a serving size between six and twelve ounces and
maintaining the wellness mixture refrigerated until consumption by a user.

16. A container of ingredients for a wellness drink comprising no water, the container comprising a mixture of apple cider vinegar, aloe vera, lemon juice, honey and olive oil liquids and three spices selected from cayenne pepper, ginger, cinnamon and turmeric.

17. A tea bag comprising the following ingredients, the range of amount of each ingredient being between ⅛ teaspoon and ¼ teaspoon,
turmeric,
ground ginger, and
cayenne pepper.
